(19) United States
(12) Patent Application Publication
(10) Pub. No.: US 2006/0127967 A1
(43) Pub. Date: Jun. 15, 2006

(54) In vitro test for studying compound predicting pharmacologic and/or harmacokinetic and/or pharmacodynamic parameters of a compound

(76) Inventors: Elki Touitou, Jerusalem (IL); Biana Godin, Jerusalem (IL)

Correspondence Address: PEARL COHEN ZEDEK, LLP, 1500 BROADWAY 12TH FLOOR, NEW YORK, NY, 10036, US

(21) Appl. No.: 10/532,021

(22) PCT Filed: Oct. 23, 2003
(86) PCT No.: PCT/IL03/00876

§ 371 Date: Jan. 17, 2006

Related U.S. Application Data
(60) Provisional application No. 60/420,234, filed on Oct. 23, 2002.

(51) Int. Cl. C12Q 1/18

(52) U.S. Cl. 435/32

(57) ABSTRACT

The invention provides a method and an assay for predicting the pharmacologic and/or pharmacokinetic and/or pharmacodynamic activity and/or effective concentration of a test material using cell and/or protozoa and/or micro-organism by assessing the effect of the test material on the and/or protozoa and/or micro-organism. Further, the invention provides an apparatus or a system, which comprises a donor and a receiver compartments, separated by membrane, wherein cell and/or protozoa and/or micro-organism are present in the receiver compartments, for predicting the pharmacologic of pharmacokinetic effects such as effective concentration of a test material, by assessing the effect of the test material, on the cell and/or protozoa and/or micro-organism. In addition, the invention provides use of an artificial human skin for measuring the diffusion or the penetration of a test material through the skin.

### FIELD OF THE INVENTION

The invention provides a method and an assay for predicting the pharmacologic and/or pharmacokinetic and/or pharmacodynamic activity and/or effective concentration of a test material using cell and/or protozoa and/or micro-organism by assessing the effect of the test material on the and/or protozoa and/or micro-organism. Further, the invention provides an apparatus or a system, which comprises a donor and a receiver compartments, separated by membrane, wherein cell and/or protozoa and/or micro-organism are present in the receiver compartments, for predicting the pharmacologic of pharmacokinetic effects such as effective concentration of a test material, by assessing the effect of the test material, on the cell and/or protozoa and/or micro-organism. In addition, the invention provides use of an artificial human skin for measuring the diffusion or the penetration of a test material through the skin.

### BACKGROUND OF THE INVENTION

The effectiveness of drug applied to a skin surface is dependent on the extent of percutaneous absorption or penetration. For in vitro studies, various diffusion cells have been devised. A cell, comprising a cap, a body and 0-ring, devised by Thomas J. Franz for his study reported in J. Investigative Dermatology, 64, 190 (1975) has been adapted as a commercially available Franz diffusion cell. In this device, the body of the cell constituting the receptor chamber is that portion containing a physiological solution into which a test drug diffuses or penetrates through a test membrane which oftentimes is skin, animal membranes, synthetic membranes, cultured tissue on synthetic membranes, natural membranes, artificial membranes, artificial tissues, artificial skin and the like. The skin may be dermatomed or full-thickness, animal or human skin, tissue culture, other membranes from GI tract, kidneys, blood and other vessels from human or animal source, lungs and upper respiratory tract from human or animal source. The drawback of these membranes are lack of availability, as well as lack of reproducibility huge inter- and intra-individual variation, availability and cost of safety tests (HIV, HBV, etc.).

In addition there is a need for a well-defined, reproducible source of cells available in unlimited amounts for replacing in vivo methods of detecting pharmacologic and pharmacokinetic characteristics of a drug or a test material. A similar need exists for in vitro drug screening.

Further, there is a need of simultaneously testing the ability of a compound to permeate or diffuse through a membrane and to the predict its pharmacological and/or pharmacokinetic response.

### SUMMARY OF THE INVENTION

The methods of the invention can be used to screen and design compound libraries, select and design drugs, as well as predict drug efficacy in mammals by using in vitro method. The methods of the invention also finds use in selecting, designing, and preparing drug compounds, and multi-compound drugs and drug formulations (i.e., drug delivery system) for preparation of medicaments for use in treating mammalian disorders.

In one embodiment, the invention provides a method of predicting pharmacologic and/or pharmacokinetic and/or pharmacodynamic activity of a test material comprising the steps of incubating different concentrations of the test material with cell and/or protozoa and/or micro-organism; and determining the change in the morphology of the cell and/or protozoa and/or micro-organism;

wherein the change in the morphology serves for the calculation of the effective concentration of the test material in the blood, thereby predicting pharmacologic and/or pharmacokinetic and/or pharmacodynamic activity of the test material.

In another embodiment of the invention, there is provided a method of predicting the effective concentration of a test material in the blood comprising the steps of: incubating different concentrations of the test material with cell and/or protozoa and/or macro-organism; and determining the change in the morphology of the cell and/or protozoa and/or micro-organism; wherein the change in the morphology serve for the calculation of the effective concentration of the test material in the blood, thereby predicting the effective concentration of the test material.

In another embodiment of the invention, there is provided method of predicting the plateau/maximum/steady state/peak concentration of a test material in the blood comprising the steps of: incubating different concentrations of the test material with cell and/or protozoa and/or micro-organism; and determining the change in the morphology of the cell and/or protozoa and/or micro-organism; wherein change in the morphology serve for the calculation of the effective concentration of the test material in the blood, thereby predicting the plateau/maximum/steady state/peak concentration of the test material.

In another embodiment of the invention, there is provided a method of selecting a composition among a plurality of compositions, which comprise the same active ingredient, so as to obtain an effective concentration of the active ingredient in the blood, comprising the steps of: incubating at least one dose of each composition with cell and/or protozoa and/or micro-organism; determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment of the invention, there is provided a method of selecting a composition among a plurality of compositions, which comprise the same active ingredient, so as to obtain plateau/maximum/steady state/peak concentration of the active ingredient in the blood, comprising the steps of: incubating at least one dose of each composition with cell and/or protozoa and/or micro-organism; determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment of the invention, there is provided a method of predicting the effective concentration of a drug in the blood comprising the steps of: incubating different concentrations of the drug with Tetrahymena species and/or other cultured cells; determining the difference in the proliferation rate of the Tetrahymena species and/or other cultured cells; wherein the proliferative effect serve for the calculation of the effective concentration of the test material in the blood, thereby predicting the effective concentration of a drug.

In another embodiment of the invention, there is provided a method of selecting a composition among a plurality of compositions, which comprise the same active ingredient, so as to obtain an effective concentration of the active ingredient in the blood, comprising the steps of: incubating at least one dose of each composition with Tetrahymena species and/or other cultured cells; determining the difference in the proliferation of the Tetrahymena species and/or other cultured cells, and comparing the difference elicit by each composition, wherein a composition which causes higher difference in proliferation will have higher concentration of the active ingredient in the blood; thereby selecting a composition among a plurality of compositions, which comprise the same active ingredient, so as to obtain an effective concentration of the active ingredient in the blood.

In another embodiment of the invention, the protozoa is Tetrahymena pyriformis, Tetrahymena thermophila.

In another embodiment of the invention, there is provided the method may further comprising a step of comparing the morphological effect on the cell and/or protozoa and/or micro-organism and the pharmacological effect of at least two known drugs of the family of drugs to which the test material belong to, so as to predict the blood concentration of the test material.

In another embodiment of the invention, there is provided an apparatus comprising a) a donor compartment for retaining a sample of test material to be tested for extent of diffusion and/or permeation through a test membrane; and (b) a receiver compartment, which comprises cells and/or protozoa and/or micro-organisms, wherein the test membrane is located between the donor compartment and the receiver compartment.

In another embodiment of the invention, there is provided a method of predicting pharmacologic and/or pharmacokinetic and/or pharmacodynamic activity of a test material comprising the steps of: administering to the donor compartment according to the invention, a sample of the test material and determining the difference in the morphology caused by the test material, on the cells and/or protozoa and/or micro-organisms, wherein the morphological difference serves for the predicting pharmacologic and/or pharmacokinetic and/or pharmacodynamic activity.

In another embodiment of the invention, there is provided a method of predicting the effective concentration of a test material in the blood comprising the steps of: administering to the donor compartment according to the invention, a sample of the test material; and determining the change in the morphology of the cell and/or protozoa and/or micro-organism; wherein change in the morphology serve for the calculation of the effective concentration of the test material in the blood, thereby predicting the effective concentration of test material.

In another embodiment of the invention, there is provided a method of predicting the sub-minimum, plateau/maximum/steady state/peak concentration of a test material in the blood comprising the steps of: administering to the donor compartment according to the invention, a sample of the test material; and determining the change in the morphology of the cell and/or protozoa and/or micro-organism; wherein change in the morphology serve for the calculation of the effective concentration of the test material in the blood, thereby predicting the sub-minimum, plateau/maximum/steady state/peak concentration of test material.

In another embodiment of the invention, there is provided a method of selecting a transdermal composition among a plurality of compositions, which comprise the same active ingredient, so as to obtain an effective concentration of the active ingredient in the blood, comprising the steps of: adding at least one dose of each composition to the apparatus of the invention; determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment of the invention, there is provided a method of selecting a transdermal compound among a plurality of compounds, so as to obtain an effective concentration of the active ingredient in the blood, comprising the steps of: adding at least one dose of each compound to the apparatus of the invention; determining the difference in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment of the invention, there is provided a method of predicting the effective concentration of a drug in the blood comprising the steps of: adding at least one dose of each compound to the apparatus of the invention; and determining the difference in the proliferation rate of the cell and/or protozoa and/or micro-organism; wherein the proliferative rate serves for the calculation of the effective concentration of the test material in the blood, thereby predicting the effective concentration of a drug.

In another embodiment of the invention, there is provided a method of selecting a composition among a plurality of compositions, which comprise the same active ingredient, so as to obtain an effective concentration of the active ingredient in the blood, comprising the steps of: adding at least one dose of each composition to the apparatus of the invention; determining the difference in the proliferation of the cell and/or protozoa and/or micro-organism so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment of the invention, there is provided use of an apparatus comprising a bi-phasic membrane comprising of silicon and collagen for measuring permeation or diffusion of a test material through a membrane.

In another embodiment of the invention, there is provided use of bi-phasic membrane comprising of silicon and collagen for measuring permeation or diffusion of a test material through a membrane.

### BRIEF DESCRIPTION OF THE DRAWINGS

FIGS. 1-4 demonstrate a schematic view of an apparatus according to an embodiment of the invention.

FIG. 5 demonstrates a multiple cell assembly schematic view of an apparatus according to an embodiment of the invention.

FIG. 6 demonstrates a three compartmental cell schematic view of an apparatus according to an embodiment of the invention.

FIG. 7 (a and b) demonstrate a schematic view of a single chain (a) and a multiple assembly (b) according to an embodiment of the invention.

FIG. 8 (a and b) demonstrate a schematic view of multiple assembly according to an embodiment of the invention.

FIG. 9 (a and b) demonstrate a schematic view a single chain (a) and a multiple assembly (b) according to an embodiment of the invention.

FIG. 10 (a and b) demonstrates a schematic view of multiple assembly according to an embodiment of the invention.

FIG. 11 demonstrates a schematic view of an apparatus according to an embodiment of the invention, wherein cells are included in the receiver compartment.

FIG. 12 (a and b) demonstrates morphological changes in Tetrahymena thermophila which are in correlation to changes in drug (sodium salicylate) concentration.

FIG. 13 (a and b) demonstrates morphological changes in Tetrahymena thermophila which are in correlation to changes in drug (alprenolol hydrochloride) concentration.

FIG. 14 demonstrates morphological changes in Tetrahymena thermophila which are in correlation to changes in different drugs concentration.

FIG. 15 demonstrates propranolol permeation at pH 7.2 through human skin vs. Integra.

FIG. 16 demonstrates correlation between Tetrahymena cell area and effective plasma anti-inflammatory drugs concentration.

FIG. 16 demonstrates correlation between Tetrahymena cell area and effective plasma anti-inflammatory drugs concentration.

FIG. 17 demonstrates a scheme for the method and apparatus for simultaneaously measuring the permeation and diffusion for skin and their effects on cells morphology.

### ABBREVIATIONS

M/T/Sk=Membrane/Tissue/Skin

R=Receiver

D/Dr=Donor/Drug

TR=tetrahymena species and/or other cells

### DESCRIPTION OF THE DETAILED EMBODIMENTS

The invention provides a method and an assay for predicting the effective concentration of a test material using cell and/or protozoa and/or micro-organism by assessing the effect of the test material on the cell and/or protozoa and/or micro-organism. Further, the invention provides an apparatus, system and method for predicting the effective concentration of a test material, by assessing the effect of the test material, administered to an apparatus or system, which comprise a donor and a receiver compartments, separated by membrane, on cell and/or protozoa and/or micro-organism, which are present in the receiver compartments. In addition, the invention provides use of an bi-phasic mamebrane for measuring and predicting the diffusion or the penetration of a test material through the skin. The methods and bioassays disclosed in the invention may be used to substitute in vivo bioassays such as vasoconstrictor, anesthesia, analgesia, blanching, redness, anti-inflammation, inflammation, immunomudolation assay in humans, bioavailability and/or toxicity human studies for the development and test of dermal or transdermal pharmaceutical and/or cosmetic products.

The test material can be further developed into a drug. In another embodiment, the test material is a lead compound. In another embodiment, the test material is a chemical entity. The term “drug” can refer to any pharmaceutically active substance capable of being administered in a particulate formulation, which achieves the desired effect. Drugs can be synthetic or natural organic compounds, proteins or peptides, oligonucleotides or nucleotides, or polysaccharides or sugars. Drugs may have any of a variety of activities, which may be inhibitory or stimulatory, such as antibiotic activity, antiviral activity, antifungal activity, steroidal activity, cytotoxic or anti-proliferative activity, anti-inflammatory activity, analgesic or anesthetic activity, hormones, anti-mitotic, erectiles, sleep inducers, anti-depressants, cancer agents, anti-histamine agents, anti-allergic, or be useful as contrast or other diagnostic agents. A description of classes of drugs and species within each class can be found in Martindale, The Extra Pharmacopoeia, 31st Ed., The Pharmaceutical Press, London (1996) and Goodman and Gilman, The Pharmacological Basis of Therapeutics, (9th Ed., McGraw-Hill Publishing company (1996).

In one embodiment, the invention provides a method of predicting pharmacologic and/or pharmacokinetic and/or pharmacodynamic activity and/or pharmacodynamic efficiency of a test material comprising the steps of: incubating different concentrations of the test material with cell and/or protozoa and/or micro-organism; and determining the difference in the morphology of the cell and/or protozoa and/or micro-organism;

wherein said change in the morphology serves for the calculation of the effective concentration of the test material in the blood, thereby predicting pharmacologic and/or pharmacokinetic and/or pharmacodynamic activity of the test material.

In one embodiment, the pharmacologic and/or pharmacokinetic and/or pharmacodynamic activity is referred in the invention to toxicity and/or metabolism and/or distribution and/or elimination of the test material, and combination thereof.

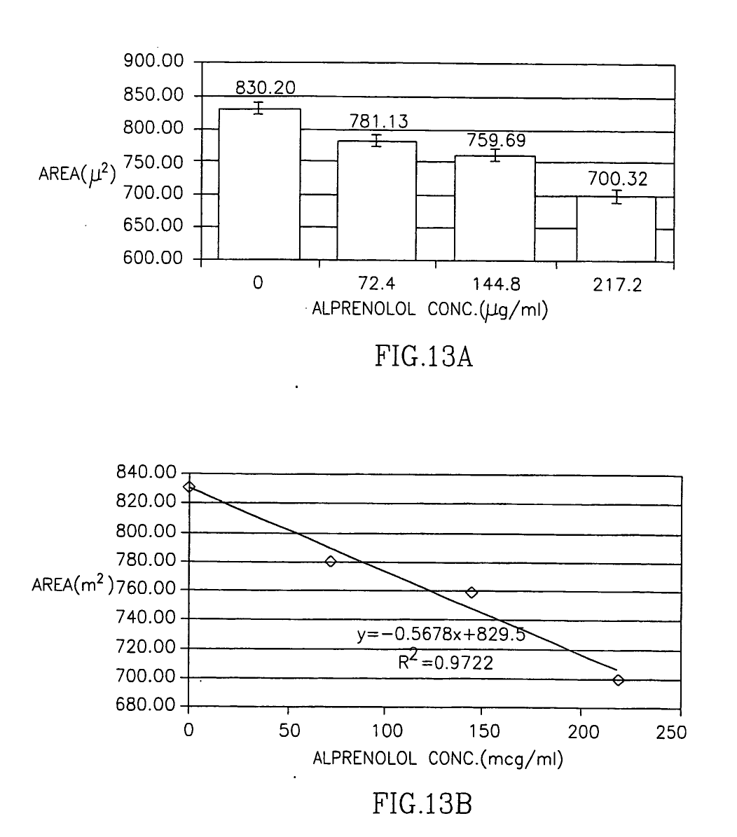

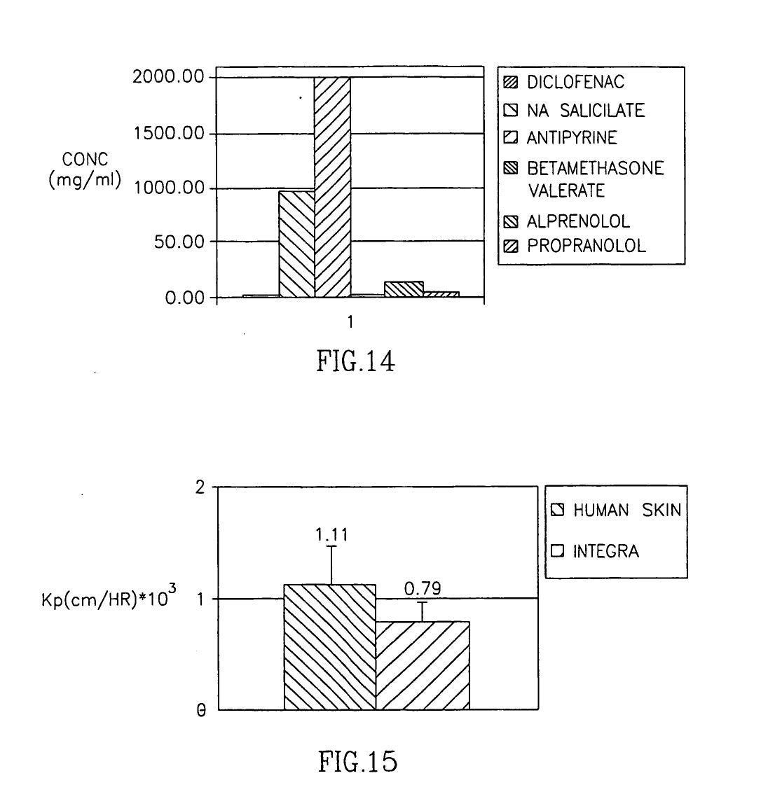

Specifically, the invention shows unexpected correlation reflected by the slope of the linear regression between drug concentration and Tetrahymena area. An example of such unexpected correlation is provided of FIGS. 13 and 14. As a test organism, Tetrahymena presents important advantages. This organism represents cellular structure and functional complexity comparable with those of human cells. A high degree of matching between Tetrahymena and human genes found encouraged the use of these protozoa in functional genome research at the cellular level. Tetrahymena is characterized in a short life cycle, easy cultivation in various growth conditions. Tetrahymena was the first protozoan to be cultivated in axenic conditions and in a chemically defined medium.

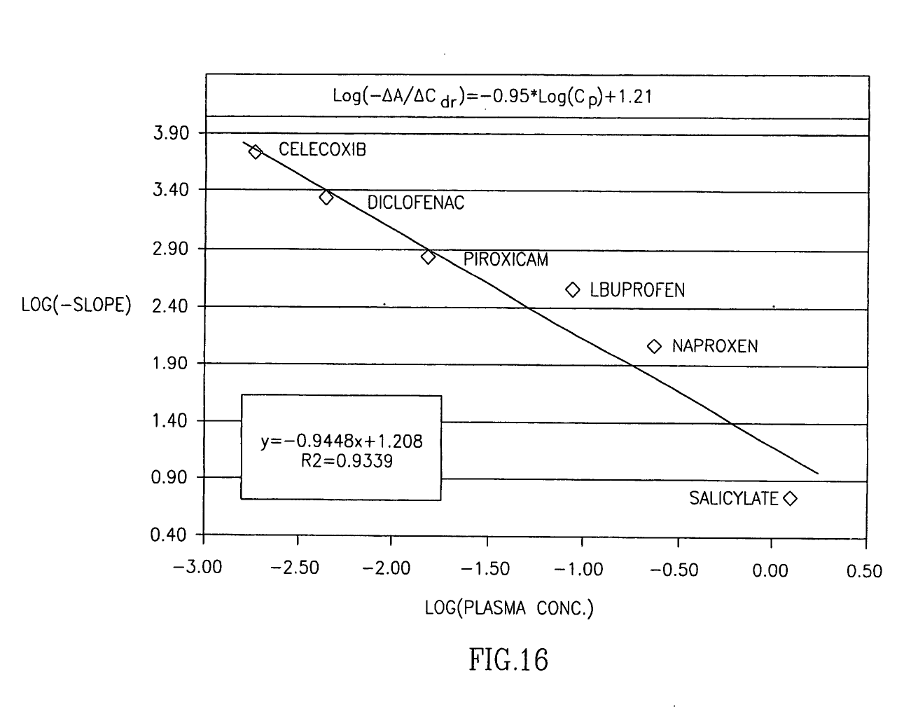

This correlation as can be seen in FIG. 16 is specific for each pharmacological group of drugs.

This correlation can be use to predict effective drug concentration in blood (efficacy) for new entities or for molecules for which this parameter is not known. Further, sub-minimum, plateau/maximum/steady state/peak concentration may be predicted by correlation to Tetrahymena area. This correlation can be used as a predictive tool for lead compounds and/or ADME/T (absorption, distribution, metabolism, elimination toxicity) of new molecules and/or instead of bioavailability studies and/or efficacy studies and/or in pharmacological research and/or pharmacodynamic studies and/or pharmacokinetic studies, which are conducted in animals or in human being and are time consuming and sometimes lack reproducibility.

In another embodiment, the prediction of the concentration is predicted in other body fluids such as the urine an the CSF.

In one embodiment, effective dose may be dose of a drug predicted (by statistical techniques) to produce a characteristic effect in 50 percent of the subjects to whom the dose is given. The median effective dose (usually abbreviated ED50) is found by interpolation from a dose-effect curve. The ED50 is the most frequently used standardized dose by means of which the potencies of drugs are compared. However, one can determine the dose of drug predicted to be effective in one percent (ED1) or 99 percent (ED99) of a population.

It also can be used as a parameter (descriptor) for in Silico design.

The relevant data may be transferred to a computer software and/or program so as to process the data.

The cell or protozoa, which may be used in the invention are in one embodiment, Tetrahymena pyriformis or Tetrahymena termophila.

Other cells which may be used in the invention may animal or human cultured cells grown in dispersion, plant, animal or human cultured cells grown on plates (as monolayer), bacteria, etc.

Examples of plant, animal or human cultured cells grown in dispersion: eosinophils, mast cells, langerhanz cells, eryhtrocytes etc.

In another embodiment of the invention bacteria may be used in the method/bioassay of the invention, such as, for example: species of Staphylococcus, Streptococcus, Vibrio, Bacillus, etc.

In an embodiment of the invention, the cells may be grown in dispersion or on plates as mono-layers or multi-layers.

In one embodiment, the difference in morphology is a difference in area of the cell/protozoa or micro-organism. In another embodiment, the difference is a difference in shape factor. In another embodiment, the difference in morphology is a difference in the volume of the cells. In another embodiment, the difference in morphology is calculated by difference in radius, perimeter or the diameter of the cell and/or protozoa and/or micro-organism.

The term “blood” encompasses, in an embodiment of the invention, plasma, serum, extracellular fluid or lymph fluid.

In one embodiment of the invention, “effective concentration of a test material” is a concentration of the active ingredient or ingredients, which elicit a therapeutic and/or diagnostic pharmacological effect.

In an embodiment of the invention, “the calculation of the effective concentration of the test material in the blood” may be done as follows:

The slope of the linear regression between concentration in the blood (which may be either effective or sub-minimum, plateau/maximum/steady state/peak concentration) of at least two known drugs and a concentration dependent changes in Tetrahymena area, radius, shape and other parameters are calculated. As is shown in FIG. 16, compounds from the same group have a correlation between the concentration in blood and the concentration dependent change in Tetrahymnea area. The value of the difference in the area of Tetrahymena of the unknown test material is then added to the curve and the concentration in the blood is predicted. The calculation can be done by using computerized software. The change in morphology may be evaluated by image analysis, computerized image analysis, morphometric program or morphometric bioassay.

The term “active compound” or “active ingredient” means a compound, which is either a pharmaceutically or pharmacologically active drug or agent, or a detectably labeled compound. In turn, a “pharmaceutically or pharmacologically active drug or agent” shall be interpreted to mean any pharmaceutically effective compound used in the treatment or diagnosis of disease.

In one embodiment of the invention, the correlation is specific for each pharmacological group of drugs.

In another embodiment, the correlation is specific for drugs or test material, which are acting on similar receptor.

In another embodiment of the invention, the correlation is specific for drugs of the (non-inflammatory NSAID's group).

In another embodiment of the invention, the correlation is specific for drugs of the beta blockers group.

This correlation can be use to predict effective drug concentration in blood (efficacy) for new entities or for molecules for which this parameter is not known.

Accordingly, the invention provides, in one embodiment, a method of predicting the effective concentration of a test material in the blood comprising the steps of:

incubating different concentrations of the test material with cell and/or protozoa and/or micro-organism; and determining the change in the morphology of the cell and/or protozoa and/or micro-organism; wherein said change in the morphology serve for the calculation of the effective concentration of the test material in the blood, thereby predicting the effective concentration of the test material.

As was noted before, in one embodiment, the invention provides a method of predicting the plateau/maximum/steady state/peak concentration of a test material in the blood comprising the steps of: incubating different concentrations of the test material with cell and/or protozoa and/or micro-organism; and determining the change in the morphology of the cell and/or protozoa and/or micro-organism; wherein change in the morphology serves for the calculation of the effective concentration of the test material in the blood, thereby predicting the sub-minimum, plateau/maximum/steady state/peak concentration of the test material.

In another embodiment of the invention, the invention may be used as a method of selecting composition with an active ingredient and at least one excipient among a plurality of compositions, which comprise the same active ingredient and other excipient or excipients, so as to obtain an effective concentration of the active ingredient in the blood, comprising the steps of: incubating at least one dose of each composition with cell and/or protozoa and/or micro-organism; determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood. According to this embodiment, the optimal composition may be selected for obtaining the ddesired effect on blood concentration.

The drug substance may be “associated” in any physical form with a particulate material, for example, adsorbed or absorbed, adhered to or dispersed or suspended in such matter, which may take the form of discrete particles or microparticles such as liposomes, vesicles, lipospheres, microspheres, micro-capsules, matrix, adhesive in any medicinal preparation, and/or suspended or dissolved in a carrier such as an ointment, gel, paste, lotion, sponge, cream, suspension or spray.

Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, poly-oxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, hyaluronic acids and hyaluronate, penetration retardants, penetration enhancers, acrylate, adhesives, sugars and starches

Similarly, in another embodiment of the invention, the invention may be used as a method of selecting a composition with an active ingredient and at least one excipient among a plurality of compositions, which comprise the same active ingredient, so as to obtain plateau/maximum/steady state/peak concentration of the active ingredient in the blood, comprising the steps of: incubating at least one dose of each composition with cell and/or protozoa and/or micro-organism; determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment, the invention provides a method of predicting the effective concentration of a drug in the blood comprising the steps of: incubating different concentrations of the drug with Tetrahymena species and/or other cultured cells; determining the difference in the proliferation rate of the Tetrahymena species and/or other cultured cells; wherein said proliferative effect serve for the calculation of the effective concentration of the test material in the blood, thereby predicting the effective concentration of a drug.

In another embodiment, the invention provides a method of selecting a composition among a plurality of compositions, which comprise the same active ingredient, so as to obtain an effective concentration of the active ingredient in the blood, comprising the steps of: incubating at least one dose of each composition with Tetrahymena species and/or other cultured cells; determining the difference in the proliferation of the Tetrahymena species and/or other cultured cells, and comparing said difference elicit by each composition, wherein a composition which causes higher difference in proliferation will have higher concentration of the active ingredient in the blood; thereby selecting a composition among a plurality of compositions, which comprise the same active ingredient, so as to obtain an effective concentration of the active ingredient in the blood.

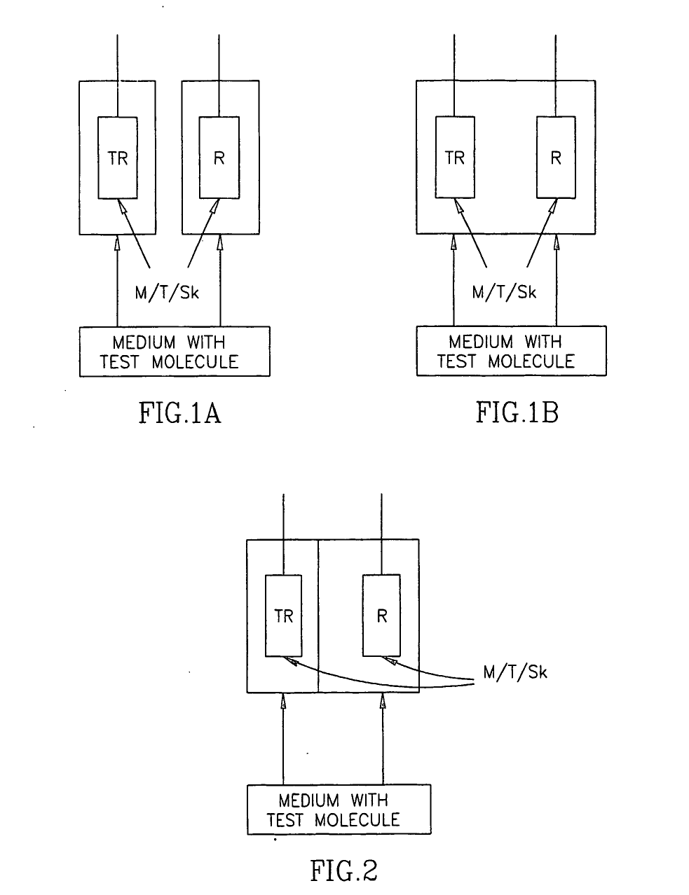

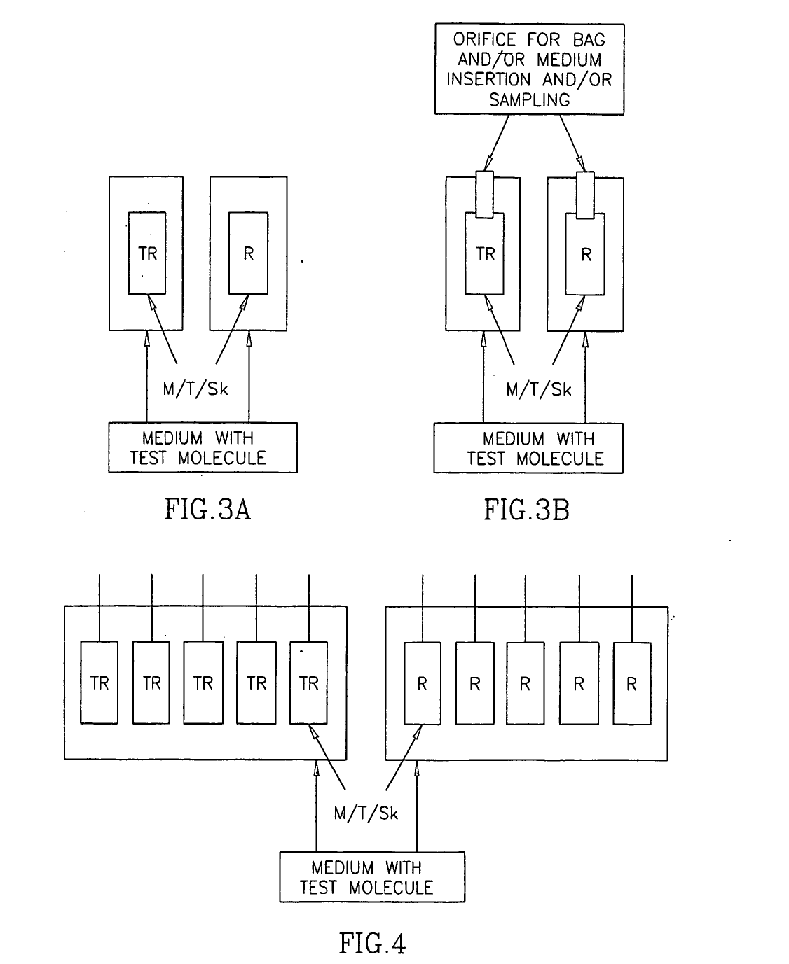

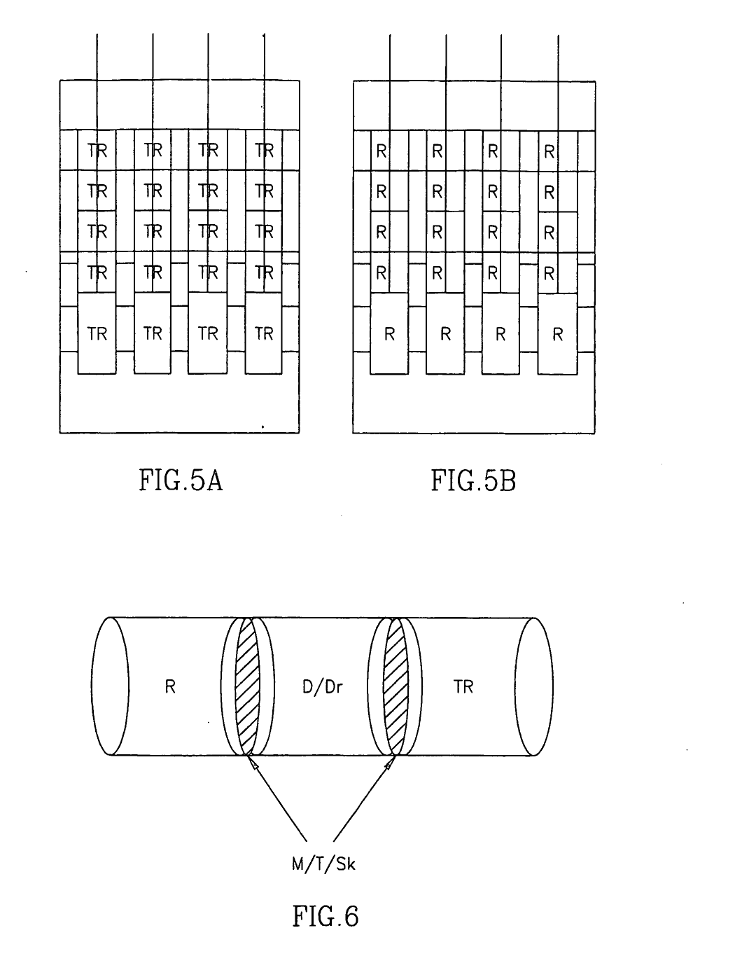

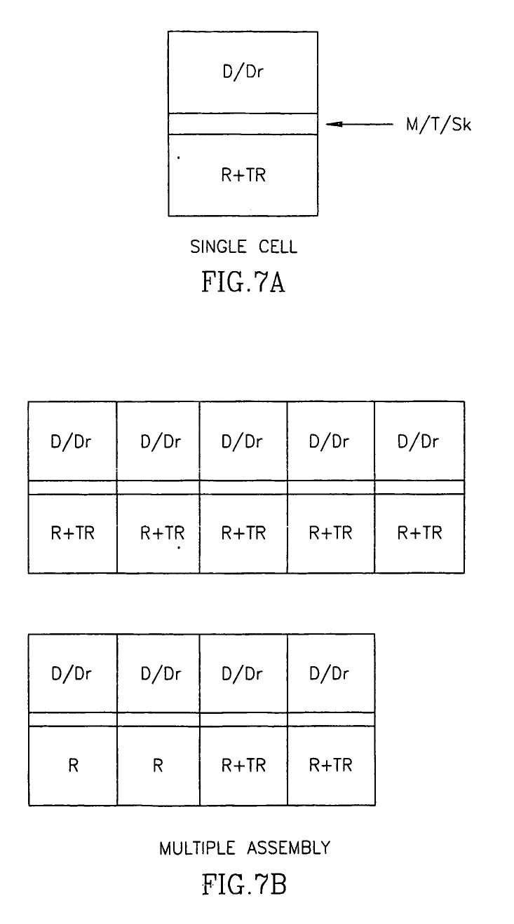

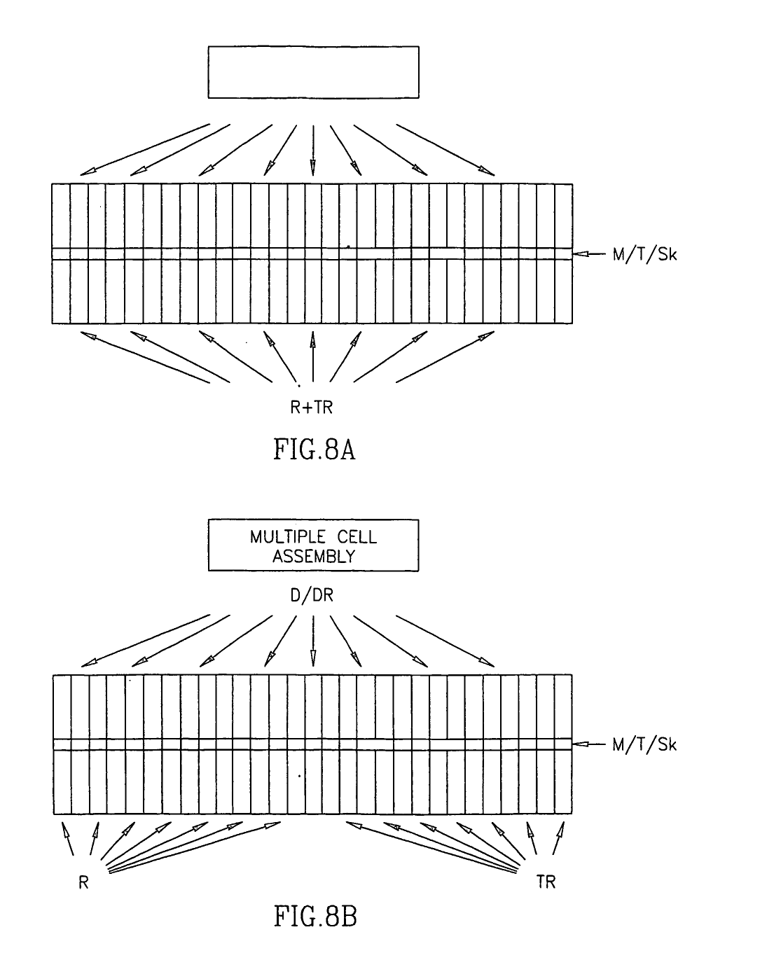

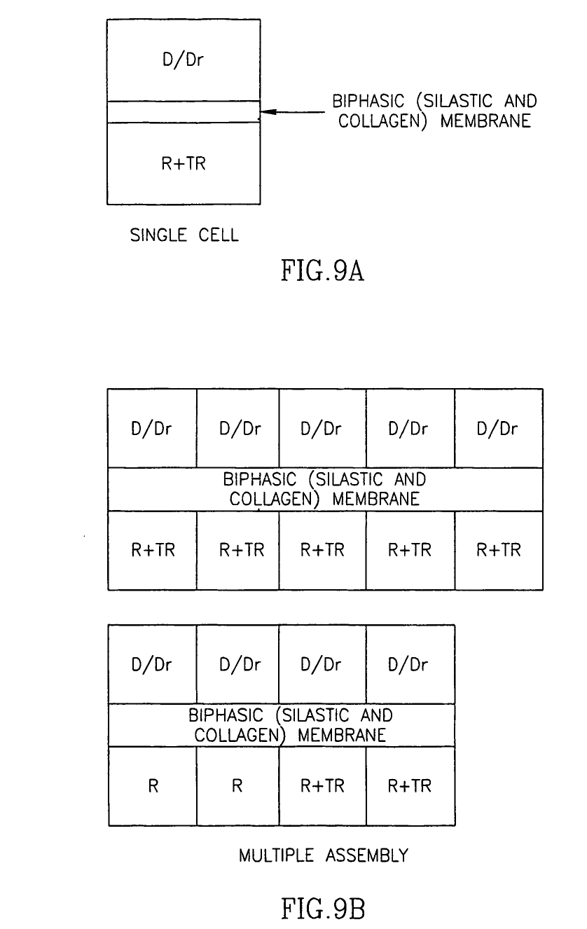

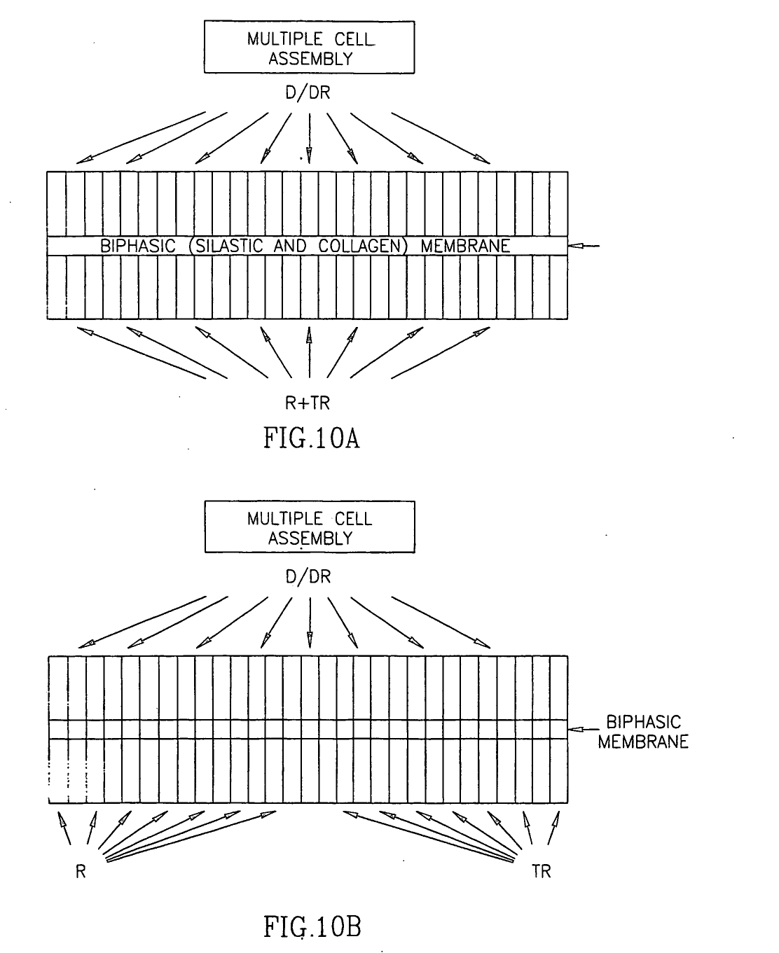

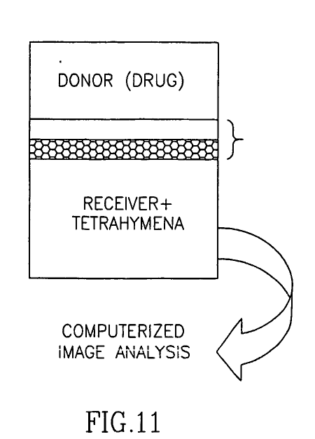

In another embodiment, the invention provides an apparatus or a device or an assembly or system which comprises more than one apparatus or device, which may be shared or separated, for simultaneously testing permeation and/or diffusion through a membrane together with a pharmacokinetic or pharmacologic activity. Various examples are presented, without limitation, on FIGS. 1-10.

“Permeability” or “permeation” are referred, in one embodiment of the invention, to the ability of a physiological barrier to permit passage of a substance. Refers to the concentration-dependent or concentration-independent rate of transport (flux), and collectively reflects the effects of characteristics such as molecular size, charge, partition coefficient and stability of a compound on transport. Permeability is substance and barrier specific.

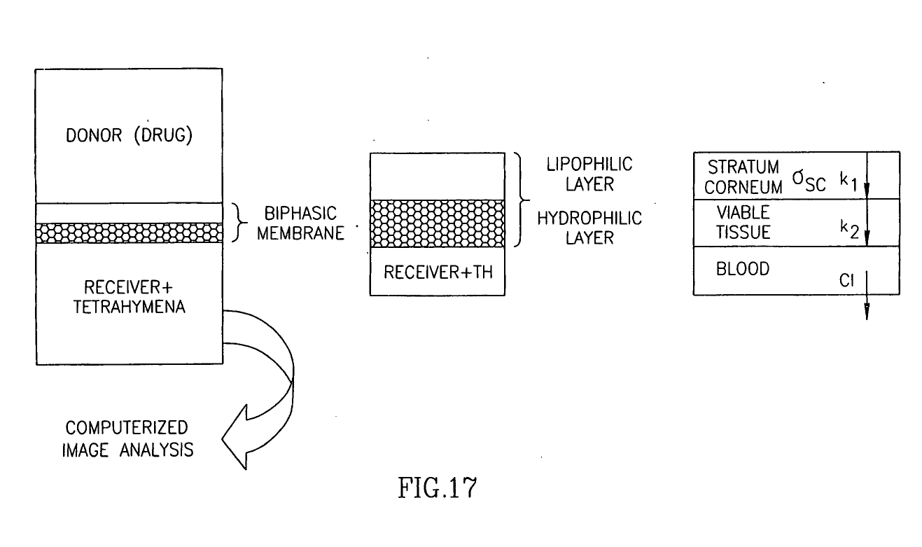

According to one embodiment of the invention, the apparatus or device comprising a) a donor compartment for retaining a sample of test material to be tested for extent of diffusion and/or permeation through a test membrane; and (b) a receiver compartment, which comprises cells and/or protozoa and/or micro-organisms, wherein said test membrane is located between said donor compartment and said receiver compartment. An example is provided in FIG. 17.

According to another embodiment, the cells and/or protozoa and/or micro-organisms are groups of any species of the groups of Tetrahymena pyriformis or Tetrahymena thermophila, Tetrahymena Borealis, Tetrahymena Americanis.

According to another embodiment the cells may be any of the cells described above.

According to an embodiment of the invention, the test membrane is a biphasic membrane possessing hydrophobic and hydrophilic layers.

According to an embodiment of the invention, the test membrane used in the invention is from natural, synthetic or semi-synthetic source.

According to an embodiment of the invention, the test membrane is animal tissue, human tissue, plant tissue, cultured collagen on silicone membrane.

According to an embodiment of the invention, the biphasic membrane comprises, in an embodiment of the invention, of silastic and/or silicone and collagen.

According to an embodiment of the invention, the hydrophobic layer is comprised of silicone.

According to an embodiment of the invention, the hydrophobic layer is comprised of collagen and/or glycosamynoglycan.

According to an embodiment of the invention, the hydrophobic layer is comprised of silicone and the hydrophobic layer is comprised of collagen and glycosamynoglycan.

According to an embodiment of the invention, the hydrophilic layer is comprised of at least one of the following components: collagen, elastin, fibrin, cell culture, synthetic hydrophilic materials, hydrophilic polymers, glycosamynoglycan, proteins or combination thereof.

The glycosaminoglycan may be selected from selected from the group consisting of chondroitin 6-sulfate, chondroitin 4-sulfate, heparin, heparan sulfate, keratan sulfate, dermatan sulfate, chitin and chitosan.

According to an embodiment of the invention, the hydrophobic layer is comprised of a one of the following components: silastic, silicone, ceramides, cholesterol, cholesteryl esters, cholesterol derivatives, phospholipids, free fatty acids, esters of free fatty acids, cellulose acetate/nitrate membrane, pure cellulose acetate with/without wetting agent, polysulfone membrane, glass fiber, Teflon, or combination thereof.

According to an embodiment of the invention, the thickness of the hydrophilic part is 0.005-3 mm.

According to an embodiment of the invention, the thickness of the lypophylic layer is 0.005-1 mm.

According to an embodiment of the invention, the thickness of the hydrophilic part is 0.05-3 mm.

According to an embodiment of the invention, the thickness of the lypophylic layer is 0.005-0.25 mm.

According to an embodiment of the invention, the apparatus or device of the invention may be in the form of sacks and/or “teabags” and/or tubes and/or pockets and/or plates, dishes and/or containers. They may be modified or unmodified side-by-side (Valia-Chien) diffusion cells and/or modified or unmodified Franz cells and/or modified or not modified flow-through cells (such as Brounogh cells) and/or multi-well plates that contain and/or contact the tested molecule and/or drug and/or chemicals etc.

In an embodiment of the invention, the assembly may contain one and/or several compartments in one and/or several, shared and/or separated containers.

In an embodiment of the invention, the assembly may be automatically and/or manually operated.

The units or assemblies may be disposable or for multiple use.

The units or assemblies can contain other additions such as magnetic stirrers and magnets for mixing the medium, thermostats for maintaining fixed temperature, gas suppliers and others.

The compartments of the assemblies may be made from glass, Pyrex, plastic, Teflon, polymers, stainless steel, coated and/or not coated metals, ceramics, silastic, cellulose polymers (e.g. specially treated cardboard), graphite, Bakelite.

The bags (containers, etc) may be made from various natural and/or synthetic and/or semi-synthetic membranes and/or artificial skin and/or artificial tissues or contain rigid solid material, and/or semisolids, and/or tissues (eg. skin).

The assemblies such as sacks and/or teabags and/or pockets and/or containers could be placed in another bigger fixed and/or disposable container.

In an embodiment of the invention, the donor compartment may contain aqueous or other special media or formulations and products.

In an embodiment of the invention, the receiver compartment may contain aqueous or other special media.

Examples of receiver fluids are, without limitation, each of and the combinations of the following: PPYE, Tris buffer, water, aqueous media, cell growth media, aqueous solutions of polysorbates, DMSO, propylene glycol, ethanol, transcutol in various concentrations, acetate buffer, phosphate buffer, micellar solution, vesicular systems, emulsions, serum, solutions of proteins.

According to an embodiment of the invention there is provided a method of predicting pharmacologic and/or pharmacokinetic and/or pharmacodynamic activity of a test material comprising the steps of: administering to the donor compartment of the apparatus, device, assembly or system of the invention, a sample of the test material and determining the difference in the morphology caused by the test material, on the cells and/or protozoa and/or micro-organisms which are in the receiving donor wherein said morphological difference serves for the predicting pharmacologic and/or pharmacokinetic and/or pharmacodynamic activity as was explained before.

The methods of the invention which are based on the use of apparatus, device, system or assembly of the invention, which comprise cells and/or protozoa and/or micro-organism may used in an embodiment of the invention for assessing the effects of a test material or composition, which may be applied in local, dermal, transdermal or cosmetic application. The advantage of these method is that it enables both predicting the diffusion or penetration through the skin and also the pharmacologic or pharmcokinetics effect of the test.

The formulations may be administered, in one embodiment of the invention, locally within the region to be treated, for example, vaginally for treatment of diseases of the ovaries and uterus. As used herein, “locally” can refer to topical application generally to the mucosal or endometrial surfaces of the vagina and/or uterus, or to a particular portion of the vagina or uterus. As used in “trandermally” refers to administration via the skin, wherein the active ingredient is diffuses and/or permeate via the skin, to the circulatory system. As used herein, “systemically” refers to the circulatory system, and regions outside the spaces described above.

In another embodiment, the invention provides a method of predicting the effective concentration of a test material in the blood comprising the steps of: administering to the donor compartment of the apparatus, device, assembly or system of the invention a sample of the test material; and determining the change in the morphology of the cell and/or protozoa and/or micro-organism; wherein change in the morphology serves for the calculation of the effective concentration of the test material in the blood, thereby predicting the effective concentration of test material.

In another embodiment the invention provides a method of predicting the sub-minimum, plateau/maximum/steady state/peak concentration of a test material in the blood or in other body fluids such as urine, CSF, comprising the steps of: administering to the donor compartment of the apparatus, device, assembly or system of the invention, a sample of the test material; and determining the change in the morphology of said cell and/or protozoa and/or micro-organism; wherein change in the morphology serve for the calculation of the effective concentration of the test material in the blood, thereby predicting the sub-minimum, plateau/maximum/steady state/peak concentration of test material.

The maximum or “peak” concentration (Cₘₐₓ) of a drug observed after its administration; the minimum or “trough” concentration (Cₘᵢₙ) of a drug observed after its administration and just prior to the administration of a subsequent dose.

Steady State concentration may be in an embodiment of the invention, the concentration of a drug or chemical in a body fluid—usually plasma—at the time a “steady state” has been achieved, and rates of drug administration and drug elimination are equal.

In another embodiment, the invention provides a method of selecting a transdermal composition among a plurality of compositions, which comprise the same active ingredient, so as to obtain an effective concentration of the active ingredient in the blood, comprising the steps of: adding at least one dose of each composition to the of the apparatus, device, assembly or system of the invention; determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment, the invention provides a method of selecting a or trandermal compound among a plurality of compounds, so as to obtain an effective concentration of the active ingredient in the blood, comprising the steps of: adding at least one dose of each compound to the apparatus, device, assembly or system of the invention; determining the difference in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In another embodiment, the invention provides a method of predicting the effective concentration of a drug in the blood comprising the steps of: adding at least one dose of each compound to the apparatus, device, assembly or system of the invention; and determining the difference in the proliferation rate of said cell and/or protozoa and/or micro-organism; wherein said proliferative rate serves for the calculation of the effective concentration of the test material in the blood, thereby predicting the effective concentration of a drug.

In another embodiment, the invention provides a of selecting a composition among a plurality of compositions, which comprise the same active ingredient, so as to obtain an effective concentration of the active ingredient in the blood, comprising the steps of: adding at least one dose of each composition to the apparatus, device, assembly or system of the invention; determining the difference in the proliferation of said cell and/or protozoa and/or micro-organism so as to select a composition, which is capable of providing an effective concentration of the active ingredient in the blood, thereby selecting a composition among a plurality of compositions, which comprise the same active ingredient, so as to obtain an effective concentration of the active ingredient in the blood.

In an embodiment of the invention, there is provided use of an apparatus comprising an biphasic membrane for more reliably and reproducibly measuring the extent of diffusion or penetration of a test material across the membrane.

Surprisingly, it was found that biphasic membrane which comprises of silastic and collagen, which is used for covering burns during their recovery could be used for skin permeation studies. This membrane was never used for such a purpose before and it was not intended for measuring permeation. In contrary, the silastic part of the membrane is claimed to provide an effective coverage of the burns that does not allow penetration of exogenous organic materials. Such an biphasic membrane is Integra (Johnson&Johnson).

As can be seen from FIG. 15, propranolol permeation at pH 7.2 through human skin was similar to the permeation through integra.

In another embodiment of the invention, there is provided use of an apparatus comprising a biphasic membrane according to the embodiments of the invention which comprises silicon and collagen for measuring permeation or diffusion of a test material through a membrane.

In another embodiment of the invention, there is provided use of bi-phasic membrane comprising of silicon and collagen for measuring permeation or diffusion of a test material through a membrane.

The objects and advantages of this invention will be more readily apparent from the following description and accompanying drawing which illustrates an embodiment of the present invention.

### EXAMPLES

### Experimental Procedure

Tetrahymena thermophila was grown axenically at 34 ?C in proteosepeptone yeast salts (PPYS) complex medium. Test cultures were prepared by inoculating the protozoa from a stock culture into PPYS to produce initial culture of ˜5000 cell/ml. The cell cultures were then grown for 24 hours at 34 ?C. In the beginning of the experiment the culture was transferred into 3 ml test tubes and exposed to the various concentrations of the tested material. Samples were withdrawn 6 hours following the exposure to the tested material. The samples fixed with formaldehyde solution were placed on the hemocytometer, observed under microscope and the area of tetrahymena was evaluated by computerized morphometric system/image analysis program. For each sample at least 100 cells were evaluated.

Experimental Results

### Example 1

### Morphological Changes (Cell Area) in Tetrahymena Thermophila Due to Changes in Sodium Salicylate Concentration

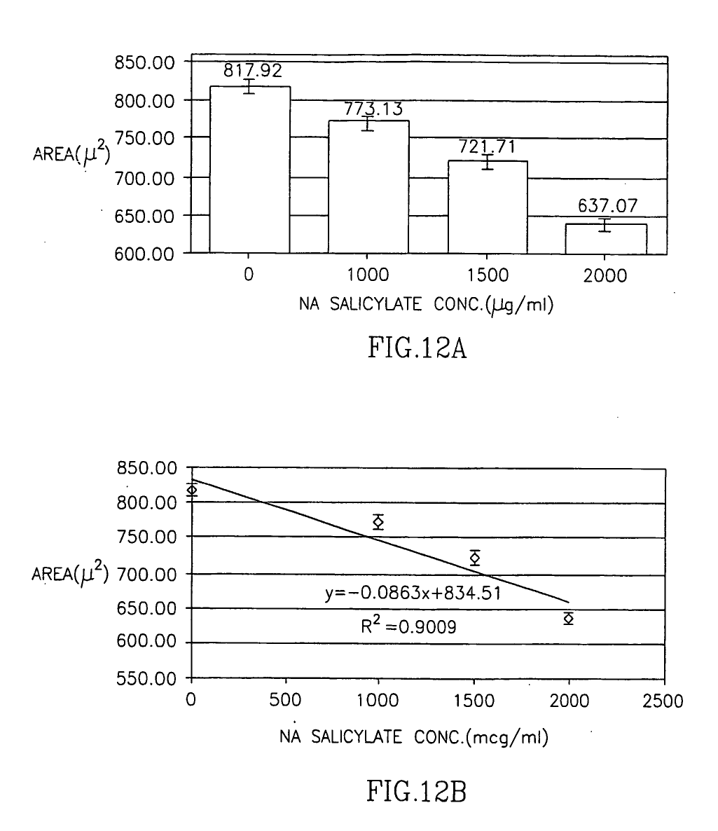

The effect of various concentrations of anti-inflammatory drug sodium salicylate at concentrations shown in FIG. 12 on the area of Tetrahymena thermophila was evaluated. As can be seen from FIG. 12, a decrease in cell area was observed when sodium salicylate concentration in the medium was higher.

The equation expresses the change in Tetrahymena thermophila area vs. sodium salicylate concentration: y=−0.0863x+834.51

### Example 2

### Morphological Changes (Cell Area) in Tetrahymena Thermophila Due to Changes in Alprenolol Hydrochloride Concentration

The effect of various concentrations of anti-inflammatory drug Alprenolol hydrochloride on the area of Tetrahymena thermophila was evaluated. As can be seen from FIG. 13, a decrease in the cell area was observed with the increase in Alprenolol hydrochloride concentration in the medium.

The equation expresses the change in Tetrahymena thermophila area vs. Alprenolol hydrochloride concentration:
y=−0.5678x+829.5

### Example 3

### Permeation of Propranolol HCl Through Bi-layer Membrane-Integra

Permeation studies with aqueous solution of Propranolol HCl (pH 7.2) through Integra and human skin were carried out in Valia-Chien diffusion cells. The effective permeation area was 0.64 cm². The receiver compartment contained Tris buffer (pH 7.2) and the experiment lasted for 8 hours. The samples were withdrawn at 0, 1, 2, 4, 6 and 8 hours and the quantity of propranolol HCl permeated through the membrane was analyzed by validated HPLC method.

As can be seen from FIG. 15, which presents permeation constant (Kp) values obtained in the experiments, there was a close similarity between Propranolol HCl (pH 7.2) Kp values obtained in permeation experiments through Integra as compared to human skin.

1-70. (canceled)

71) A method of predicting pharmacologic and/or pharmacokinetic and/or pharmacodynamic activity of a test material comprising the steps of:
incubating different concentrations of the test material with cell and/or protozoa and/or micro-organism; and determining the change in the morphology of the cell and/or protozoa and/or micro-organism;
wherein said change in the morphology serves for the calculation of the effective concentration of the test material in the blood, thereby predicting pharmacologic and/or pharmacokinetic and/or pharmacodynamic activity of the test material.

72) A method of predicting the effective concentration of a test material in the blood comprising the steps of:
incubating different concentrations of the test material with cell and/or protozoa and/or micro-organism; and determining the change in the morphology of the cell and/or protozoa and/or micro-organism;
wherein said change in the morphology serve for the calculation of the effective concentration of the test material in the blood, thereby predicting the effective concentration of the test material.

73) The method of claim 71, wherein said change in morphology is a change in area, shape factor, volume, radius, perimeter or the diameter of cell and/or protozoa and/or micro-organism.

74) The method of claim 71, wherein said protozoa is from the group of Tetrahymena pyriformis or Tetrahymena thermophila, Tetrahymena Borealis, Tetrahymena Americanis.

75) The method of claims 71, wherein said change in morphology is a change in area, shape factor, volume, radius, perimeter or the diameter of the cell and/or protozoa and/or micro-organisms.

76) The method of claim 71, wherein the test material is a drug, a lead compound or a chemical entity.

77) The method of claims 72, wherein said change in morphology is a change in area, shape factor, volume, radius, perimeter or the diameter of cell and/or protozoa and/or micro-organism.

78) The method of claim 72, wherein said protozoa is from the group of Tetrahymena pyriformis or Tetrahymena thermophila, Tetrahymena Borealis, Tetrahymena Americanis.

79) The method of claim 72, wherein said change in morphology is a change in area, shape factor, volume, radius, perimeter or the diameter of the cell and/or protozoa and/or micro-organisms.

80) The method of claim 72, wherein the test material is a drug, a lead compound or a chemical entity.

81) An apparatus comprising
(a) a donor compartment for retaining a sample of test material to be tested for extent of diffusion and/or permeation through a test membrane; and
(b) a receiver compartment, which comprises cells and/or protozoa and/or microorganisms, wherein said test membrane is located between said donor compartment and said receiver compartment.

82) The apparatus of claim 81, wherein said receiver compartment comprises any species of the groups of Tetrahymena pyriformis or Tetrahymena thermophila, Tetrahymena Borealis, Tetrahymena Americanis.

83) The apparatus of claim 81, wherein said test membrane is a biphasic membrane possessing hydrophobic and hydrophilic layers.

84) The apparatus according to claims 81, wherein said test membrane is from natural, synthetic or semi-synthetic source.

85) The apparatus of claim 84, wherein the membrane is collagen on silicone membrane.

86) A apparatus according to claim 81, wherein the hydrophobic layer is comprised of silicone.

87) A apparatus according to claim 81, wherein the hydrophobic layer is comprised of collagen and glycosamynoglycan.

88) A apparatus according to claim 81, wherein the hydrophobic layer is comprised of silicone and the hydrophobic layer is comprised of collagen and glycosamynoglycan.

89) A apparatus according to claim 81, wherein the hydrophobic layer is comprised of a one of the following components: silastic, silicone, ceramides, cholesterol, cholesteryl esters, cholesterol derivatives, phospholipids, free fatty acids, esters of free fatty acids, cellulose acetate/nitrate membrane, pure cellulose acetate with/without wetting agent, polysulfone membrane, glass fiber, Teflon, or combination thereof.

90) An apparatus according to claim 81 in the form sacks and/or “teabags” and/or tubes and/or pockets and/or plates, dishes and/or containers.

91) A system comprising at least one apparatus according to claim 81.

92) A method of predicting the effective concentration of a test material in the blood comprising the steps of:
administering to the donor compartment according to claim 81, a sample of the test material; and determining the change in the morphology of said cell and/or protozoa and/or micro-organism;
wherein change in the morphology serve for the calculation of the effective concentration of the test material in the blood, thereby predicting the effective concentration of test material.

93) A method of selecting a dermal or transdermal or cosmetic composition among a plurality of compositions, which comprise the same active ingredient, so as to obtain an effective concentration of the active ingredient in the blood, comprising the steps of:
adding at least one dose of each composition to the apparatus of claim 81;
determining the change in the morphology of the cell and/or protozoa and/or micro-organism; so as to select a composition, which is capable of providing an effective concentration of the active ingredient in the blood, thereby selecting a composition among a plurality of compositions, which comprise the same active ingredient, so as to obtain an effective concentration of the active ingredient in the blood.
